(19) United States
(12) Patent Application Publication
(10) Pub. No.: US 2018/0059004 A1
(43) Pub. Date: Mar. 1, 2018

(54) ANALYSIS SYSTEM WITH SPACING MEANS

(71) Applicant: MyCartis NV, Zwijnaarde / Ghent (BE)

(72) Inventors: Robin Muller, Tartegnin (CH); Jose De Gil, Ecublens (CH); Raphael Tornay, Illarsaz (CH); Matthieu Gaillard, Lausanne (CH)

(73) Assignee: MyCartis NV, Zwijnaarde / Ghent (BE)

(21) Appl. No.: 15/565,208

(22) PCT Filed: Apr. 11, 2016
(86) PCT No.: PCT/EP2016/057931

§ 371 Date: Oct. 9, 2017

(30) Foreign Application Priority Data
Apr. 10, 2015 (EP) 15163221.3

(51) Int. Cl. G01N 21/03; G01N 21/64; G01N 33/53; B01L 3/00

(52) U.S. Cl. CPC G01N 21/0303; G01N 21/645; G01N 33/5302; B01L 3/502715; B01L 2300/14; G01N 2021/6482; G01N 2201/021; B01L 2200/025; B01L 2300/1805; G01N 2021/0389

(57) ABSTRACT

An analysis system comprising a cartridge and an instrument being designed for operating the cartridge. The cartridge comprises a chamber sealed by a foil capable of protruding when the chamber is pressurized. The instrument comprises an analysis window and a detection portion designed for being crossed by a signal emitted from the chamber toward the instrument when the chamber is placed opposite the detection portion. The instrument further comprises docking means to place the chamber opposite the detection portion so that when the chamber is pressurized, the foil protrudes toward the detection portion. The analysis system is characterized in that it further comprises spacing means to ensure that, when the cartridge is docked on the instrument via the docking means and the chamber is pressurized, said spacing means ensure a gap between the foil and the detection portion.

The present invention relates to an analysis system comprising a cartridge and an instrument being designed for operating the cartridge and use thereof. The present invention also relates to spacing means and to the instrument for carrying out the analysis system.

Over the past few years, a forward thinking field of medicine focuses on personalized medicine to provide new levels of proteomics and genomics information and paves the way for new and tailored medical services. Personalized medicine aims at predicting, diagnosing or monitoring disease(s) at the level of individual contrary to the “one size fits all” approach. Generally, personalized medicine involves detection assays designed for detecting and quantifying one or more target biomarker(s) comprised in a sample from a patient.

When running a detection assay, the sample is usually comprised in an assay chamber of a cartridge which is processed by a detection device. The detection device usually comprises an observation window coupled to an optical system designed for the optical readout of the content of the assay chamber. In this respect, the assay chamber is conveniently placed opposite the observation window. When one or more target biomarker(s) is detected in the sample, one or more signal(s) is emitted from the assay chamber through the observation window toward the optical system of the detection device.

Typically, the detection of one or more target biomarker(s) is based on biochemical interactions between capture molecules and one or more target biomarker(s) allowing emission of one or more signal(s) which is detected by the optical system. For instance, the optical system can detect a fluorescent signal emitted when an antibody recognizes a target protein.

However, the applicant noticed that when the cartridge is processed with such a detection device, optical effects creating shadows on the observation window frequently occur. Said shadows significantly decrease the transmission of the signal through the observation window and the homogeneity of said signal so that the data provided by the detection assay are degraded by these optical effects.

Another example is disclosed in D1=US2013114076 and describes a device and an examination system providing means for optical examinations of a sample.

Technical features designed to avoid measurement errors are also described in the prior art D2=U.S. Pat. No. 6,552,784 which relates to a disposable optical cuvette device for spectrophotometric measurement of liquid samples.

Therefore, the existing detection device fails to provide reliable and accurate data when it comes to detecting and quantifying targets biomarkers.

The present invention aims to remedy all or part of the disadvantages mentioned above.

The present invention fulfills these objectives by providing an analysis system comprising a cartridge and an instrument being designed for operating the cartridge,

the cartridge comprising a chamber formed by a cavity in a portion of the cartridge, said chamber being sealed by a foil extending along said portion, said foil being capable of protruding when the chamber is pressurized, the instrument comprising an analysis window being transparent to electromagnetic signals, said analysis window comprising a detection portion designed for being crossed by a signal emitted from the chamber toward the instrument when the chamber is placed opposite the detection portion, the instrument further comprising docking means for docking the cartridge on the instrument in order to place the chamber opposite the detection portion so that when the chamber is pressurized, the foil protrudes toward the detection portion,

the analysis system being characterized in that it further comprises spacing means to ensure that, when the cartridge is docked on the instrument via the docking means and the chamber is pressurized, said spacing means ensure a gap between the foil and the detection portion.

The invention also relates to spacing means for carrying out the analysis system according to the present invention, wherein said spacing means comprise a shim.

Furthermore, the invention concerns an instrument for carrying out the analysis system according to the present invention.

The invention also concerns the use of an analysis system according to the present invention for detecting at least a target component.

Thus, the present invention solves the problem by providing an analysis system further comprising spacing means to ensure a gap between a foil of a cartridge and a detection portion of an instrument that operates said cartridge.

The applicant found out that the optical effects observed on the detection portion of the instrument according to the prior art are at least partially caused by the contact between the foil sealing the cartridge and the detection portion of the instrument. When the instrument operates the cartridge and the chamber is pressurised, the foil protrudes toward the detection portion and contacts said detection portion. Due to friction forces, the foil does not form a uniform layer but tends to generate the aforementioned optical effects, i.e. the shadows also called ripples.

Therefore, the applicant discovered that when the chamber is pressurized, the spacing means of the analysis system according to the present invention ensures a gap between the foil and the detection portion. Thus, the spacing means prevent any contact between the foil and the detection portion to avoid the optical effects observed in the prior art.

Moreover, the applicant discovered that with the analysis system of the prior art, when the chamber is pressurized and the foil contacts the detection portion, the number of transmission media crossed by a signal from the chamber to the instrument varies between a first point and a second point of the detection portion. Hence, with the analysis system according to the prior art:
the signal will cross “n” transmission media at the first point of the detection portion where the foil does contact the detection portion;
whereas at the second point where the foil does not contact the detection portion, there is a space between the foil and the detection portion so that the signal will cross an additional transmission media, i.e. the space, so that the transmission medium cross by the signal at said second point will be “n+1”.

Contrary to the analysis system according to the prior art, in the present invention when the chamber is pressurized, the spacing means prevent the contact between the foil and the detection portion so that the number of transmission media is constant at any point of the detection portion. Thus, the spacing means of the analysis system according to the present invention allows that the number of transmission media crossed by the signal from the chamber to the instrument is constant at any point of the detection portion.

Additionally, it has also been discovered that, with the analysis system of the prior art, when the foil contacts the detection portion on a contact point of the instrument creating the aforementioned optical effects, the transmission of the signal that crossed the detection portion at said contact point decreased (typically by 20%). In the present invention, the spacing means prevent the contact point and hence the decrease of the transmission of signal related thereof. Thus, the spacing means of the analysis system according to the present invention allows improving the sensitivity of the analysis system by detecting signal that would not have been detected by analysis system according to the prior art. In this respect, the spacing means also permit to improve the efficiency of the detection by preventing any contact point and hence the decrease in the transmission of the signal related thereof. The quantification is also improved.

According to an embodiment, the spacing means are separable from the analysis system. Thus, the spacing means can be installed on instrument of the prior art.

According to an embodiment, the electromagnetic signals are electromagnetic signals of which the wavelengths are comprised in a range between deep ultraviolet and deep infrared.

According to an embodiment, the electromagnetic signals are emitted from the chamber.

In an embodiment, the instrument further comprises a support comprising a recess shaped to accommodate the analysis window and the spacing means, said recess further comprising an opening opposite said analysis window.

In an embodiment, the chamber is designed for being pressurized up to 7 bars.

In an embodiment, the thickness of the shim is adapted to the foil and to the foil dimensions in order to be greater than the maximum deformation of the foil when the chamber is pressurized.

In an embodiment, the instrument further comprises heating element, said heating element being capable of transferring heat to the spacing means. Therefore, when the instrument operates the cartridge, a first part of the cartridge contacting the instrument can be at the same temperature than a second part of the cartridge contacting the spacing means. Thus, the variation of temperature between the first part of the cartridge and the second part of the cartridge is minimized.

According to an embodiment, when the cartridge is docked to the instrument via the docking means and the chamber is pressurized, the distance (d) between said detection portion and said foil is comprised between about 1 micrometer and about 250 micrometer, preferably between about 5 micrometer and about 100 micrometer, more preferably between about 10 micrometer and about 50 micrometer.

In an embodiment, the instrument comprises the spacing means.

In another embodiment, the spacing means cooperate with the analysis window.

According to a technical feature, the spacing means are designed for being placed opposite the analysis window.

According to an embodiment, the shim has a thermal conductivity between about 300 W/m K and about 1000 W/m K at 20° C. Thus, when the shim is heated, the heat is transferred to the part of the cartridge contacting the shim.

The spacing means, more particularly the shim, can be made or comprise(s) any material. In one embodiment, the shim is made of or comprises metal, more particularly the shim is made of or comprises copper.

In an embodiment, the shim further comprises reversible fastening means for fastening the shim to the instrument. Thus, when the shim is fastened to the fastening means, the shim is integral with the instrument. Furthermore, when the cartridge is docked to the instrument via the docking means and the shim is fastened to the instrument via the fastening means, the cartridge is integral with the shim. Moreover, the fastening means ensure the positioning of the shim with respect to the analysis window to maintain the gap between the detection portion and the foil of the cartridge when the cartridge is docked to the instrument.

In one embodiment, the fastening means comprises a first part and a second part, the first part comprising a tab extending from the perimeter of the shim, the second part comprising a hollow shaped in the instrument, so that when the tab is received in the hollow, the shim is fastened to the instrument.

According to an embodiment, the shim is designed for being placed on the analysis window.

In an embodiment, when the shim is placed opposite the analysis window, the shim and the support are coplanar meaning that the shim comes up with the support. In this embodiment, the support comprising the analysis window and the shim define a plan surface on the support. Thus, when the instrument operates the cartridge, the cartridge lays flat on the plan surface of support of the instrument. Therefore, if the cartridge is heated by the instrument, for instance via the heating element, the heat loss between the instrument and the cartridge is minimized.

In another embodiment, when the shim is placed opposite the analysis window, said shim defines a surface on the analysis window that matches with the detection portion of the analysis window.

The spacing means, more particularly the shim, can be of any shape. According to an embodiment, the shim is U shaped.

In an embodiment, the shim has a thickness between about 50 micrometer and about 250 micrometer, preferably between about 100 micrometer and about 200 micrometer, more preferably between about 130 micrometer and about 170 micrometer.

The present invention is further illustrated by the following detailed description set forth in view of the appended drawings, which represent an exemplary and explanatory embodiment of an analysis system according to the present invention:

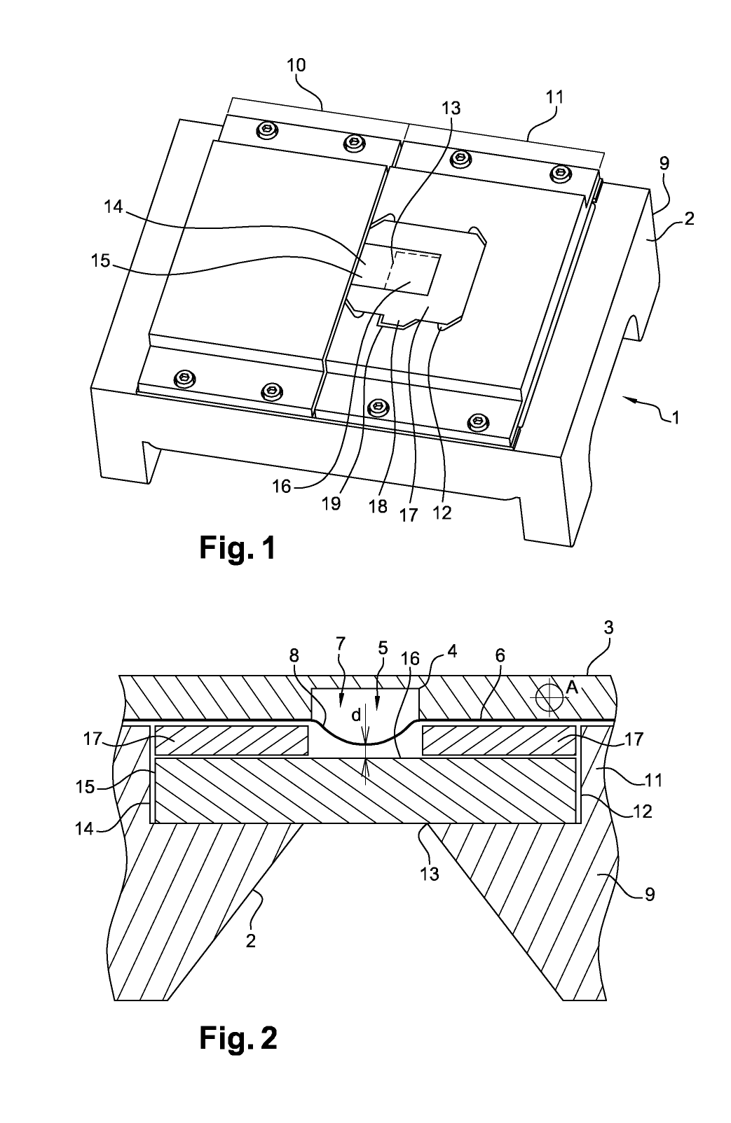

FIG. 1 illustrates a top view of an analysis system according to the present invention;

FIG. 2 illustrates a cross section view of the analysis window of the instrument of the analysis system when said instrument is operating the cartridge.

An analysis system 1 according to the present invention, partially illustrated in FIGS. 1 and 2, comprises an instrument 2 and a disposable cartridge 3 shown in FIGS. 1 and 2. The cartridge 3 and the instrument 2 are separable. The analysis system 1 according to the present invention can be used to perform an analysis of a liquid solution comprising a sample from a patient. To that end, the liquid solution is introduced in the disposable cartridge 3. Typically, the analysis aims at detecting and quantifying a biomarker or biomarkers panel from the sample in order to diagnose a disease, for instance cardiovascular disease. In this respect, the sample can be whole blood or a fractional component thereof such as plasma or serum.

The cartridge 3 comprises a chamber 4 formed by a cavity 5 in a portion 6 of the cartridge 2. In the embodiment shown in FIG. 2, the chamber 4 is formed by a channel 7 extending along the axis A of the cartridge 3. The channel 7 is connected to pressurising means (not shown in figures) designed for pressurizing the chamber 4 thereby permitting a flow of the liquid solution through the channel 7. The chamber 4 further comprises microparticles (not shown in figures) functionalized to emit electromagnetic signals toward the instrument when the targeted biomarker is detected in the sample. For instance, the surface of said microparticles can be grafted with antibodies designed for recognizing the biomarker and providing a fluorescent signal thereupon. The chamber 4 is sealed by a foil 8 extending along the portion 6 of the cartridge 3. The foil 8 is made of or comprises a deformable material allowing adjusting the volume of the chamber 4 depending on the pressure applied to the chamber 4 by the pressure means while maintaining the chamber 4 sealed. In the present embodiment, the foil 8 comprises Cyclic Olefin Polymer (COC). In the embodiment illustrated in FIG. 2, the foil 8 protrudes when the chamber 4 is pressurized.

The instrument 2 of the embodiment presented in FIGS. 1 and 2 comprises a support 9 designed for being placed opposite the cartridge 3. In the embodiment presented in FIGS. 1 and 2, the support 9 has a rectangular shaped and further comprises heating element A 10 and heating element B 11 screwed on the support 9. The instrument further comprises a recess 12, said recess further comprising an opening 13. The recess 12 is shaped to accommodate at least an analysis window 14, said analysis window 14 being placed opposite the opening 13. The analysis window 14 is transparent to electromagnetic signals and further comprises a detection portion 16 designed for being crossed by the signal emitted from the chamber 4. In the embodiment presented in FIGS. 1 and 2, the analysis window 14 is a sapphire window 15 and the detection portion 16 is designed for being crossed by the fluorescent signal emitted by the microparticle comprised in the chamber 4 upon detection of the targeted biomarkers. The instrument further comprises docking means for docking the cartridge 3 to the instrument 2. For instance, the docking means comprise a slot and a stopper (not showed in figures) so that when the cartridge 3 is docked in the docking means, the chamber 4 is placed opposite the detection portion 16. Thus, when the chamber 4 is pressurized during the assay, the foil 8 of the chamber 4 protrudes toward said detection portion 16.

The analysis system 1 further comprises spacing means to ensure that, when the cartridge 3 is docked to the instrument 2 via the docking means and the chamber 4 is pressurized, said spacing means ensure a gap between the foil 8 and the detection portion 16. For instance, in the embodiment shown in FIG. 1, the spacing means ensure that the gap, i.e. the distance (d), between the foil 8 and the detection portion 16 is at least 10 micrometer. In the embodiment shown in FIGS. 1 and 2, the spacing means comprise a shim 17, said shim 17 being shaped to surround at least partially the detection portion 16. In the present case, the shim 17 is 150 micrometer thick. In the embodiment illustrated in FIGS. 1 and 2, the shim 17 is U shaped. When the shim 17 is placed on the analysis window 14, said U shaped shim 17 defines a surface on the analysis window that matches with the detection portion 16 of the analysis window 14. The shim 17 is made of copper because copper has an appropriate thermal conductivity (385 W/(m K) at 20° C.) to ensure the heat transfer between the heating element B 11 and the cartridge 3. In the embodiment illustrated on FIG. 1, the shim 17 further comprises a tab 18 extending from the perimeter of the shim 17, said tab 18 being designed for being received in a hollow 19 of the heating element B 11 to fasten the shim 17 to the heating element B 11.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1.-6. (canceled)

7. An analysis system comprising:
a cartridge and an instrument for operating the cartridge, wherein
the cartridge comprises:
a chamber formed by a cavity in a portion of the cartridge, said chamber being sealed by a foil extending along said portion, said foil being capable of protruding when the chamber is pressurized, and
the instrument comprises:
an analysis window being transparent to electromagnetic signals, said analysis window comprising a detection portion designed for being crossed by a signal emitted from the chamber toward the instrument when the chamber is placed opposite the detection portion, and
a docking mechanism for docking the cartridge on the instrument in order to place the chamber opposite the detection portion so that when the chamber is pressurized, the foil protrudes toward the detection portion; and
a spacing mechanism to ensure that, when the cartridge is docked on the instrument via the docking mechanism and the chamber is pressurized, said spacing mechanism ensures a gap between the foil and the detection portion.

8. The analysis system according to claim 7, wherein the spacing mechanism is separable from the analysis system.

9. The analysis system according to claim 7, wherein the instrument further comprises a support comprising a recess shaped to accommodate the analysis window and the spacing mechanism, said recess further comprising an opening opposite said analysis window.

10. The analysis system according to claim 7, wherein the instrument further comprises a heating element for transferring heat to the spacing mechanism.

11. The analysis system according to claim 7, wherein when the cartridge is docked to the instrument via the docking mechanism and the chamber is pressurized, the distance between said detection portion and said foil is between about 1 micrometers and about 250 micrometers.

12. Use of an analysis system according to claim 7 for detecting at least a target component.

13. The analysis system according to claim 11, wherein the cartridge is docked to the instrument via the docking mechanism and the chamber is pressurized, the distance between said detection portion and said foil is between about 5 micrometers and about 100 micrometers.

14. The analysis system according to claim 13, wherein the cartridge is docked to the instrument via the docking mechanism and the chamber is pressurized, the distance between said detection portion and said foil is between about 10 micrometers and about 50 micrometers.
